SEQUENCE LISTING (4 sequences)

SEQ ID NO: 1 - protein, 263 aa, Homo sapiens
  Sequenza Amminoacidica della isoforma 4 della proteina umana caspasi-4
MAEGNHRKKPLKVLESLGKDFLTGVLDNLVEQNVLNWKEEEKKKYYDAKTEDKVRVMADSMQEKQRMAGQMLLQTFFNIDQISPNKKAHPNMEAGPPESGESTDALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGGEC

SEQ ID NO: 2 - protein, 377 aa, Homo sapiens
  Sequenza Amminoacidica della isoforma 1 della proteina umana caspasi-4
MAEGNHRKKPLKVLESLGKDFLTGVLDNLVEQNVLNWKEEEKKKYYDAKTEDKVRVMADSMQEKQRMAGQMLLQTFFNIDQISPNKKAHPNMEAGPPESGESTDALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGANRGELWVRDSPASLEVASSQSSENLEEDAVYKTHVEKDFIAFCSSTPHNVSWRDSTMGSIFITQLITCFQKYSWCCHLEEVFRKVQQSFETPRAKAQMPTIERLSMTRYFYLFPGN

SEQ ID NO: 3 - protein, 12 aa, Homo sapiens
  Sequenza Amminoacidica dell epitopo della sequenza della isoforma 4 della proteina umana caspasi-4 comprendente il frammento 252-263 aa.
VIIVQACRGGEC

SEQ ID NO: 4 - DNA, 15 bp, Homo sapiens
  Sequenza nucleotidica che differenzia l isoforma 4 dall isoforma 1 della proteina umana caspasi-4
ggtggtgagtgctga

(19) United States
(12) Patent Application Publication
(10) Pub. No.: US 2023/0341403 A1
(43) Pub. Date: Oct. 26, 2023

(54) METHODS AND KITS FOR THE DIAGNOSIS OF LUNG CANCER

(71) Applicants: Aldo PINTO, Napoli (NA) (IT); Rosalinda SORRENTINO, Sorrento (NA) (IT)

(72) Inventors: Aldo PINTO, Napoli (NA) (IT); Rosalinda SORRENTINO, Sorrento (NA) (IT)

(21) Appl. No.: 18/005,082

(22) PCT Filed: Jul. 14, 2021
(86) PCT No.: PCT/IB2021/056326

§ 371 Date: Jan. 11, 2023

(30) Foreign Application Priority Data
Jul. 15, 2020 (IT) 102020000017146

(51) Int. Cl. G01N 33/574; C07K 16/40; G01N 33/573

(52) U.S. Cl. CPC G01N 33/57423; C07K 16/40; G01N 33/573; G01N 2333/96466

(57) ABSTRACT

The present invention relates to a method for the diagnosis and/or to monitor the development and/or to provide a prognosis on the development of lung cancer in an individual, including the detection of the expression levels of the isoform 4 of the human caspase-4 protein in biological samples. The present invention further relates to a kit and reagents for the detection of the expression levels of said isoform 4, as well as a compound inhibitor of said isoform 4 for use in the treatment and/or prevention of lung cancer.

### FIELD OF THE INVENTION

The present invention relates a method for the diagnosis and/or to monitor the development and/or to provide a prognosis on the development of lung cancer in an individual, including the detection of the expression levels of the isoform 4 of the human caspase-4 protein in biological samples. The invention further relates to kits and reagents for the detection of the expression levels of said isoform 4, as well as to a compound inhibitor of said isoform 4 for use in the treatment and/or prevention of lung cancer.

### STATE OF ART

The lung cancer represents the third most widespread malignant neoplasm in the world and constitutes the main cause of death due to cancer in the industrialized countries, with more than a million of deaths a year.

Non-small-cell lung cancer (NSCLC), which represents about 85% of cases, is a particularly aggressive cancer form, characterized by an extremely variable clinic behaviour and by a five-year survival rate not higher than 15% as from the disease clinical finding. This dramatic condition is mainly linked to the lack of early diagnosis tools and to the recognition of symptoms only in the advanced stages of cancer. In fact, it is estimated that in about 80% of patients, at time of diagnosis, a neoplasm of metastatic origin is found. The prognosis is better if cancer is detected in an initial development step thereof, still susceptible of a radical surgical treatment, with a limited functional damage.

For these reasons, an early and correct diagnosis of lung cancer has a crucial relevance to reduce the disease impact and in particular to improve the survival possibility and life quality for cancer patients.

The diagnostic imaging techniques such as computerised tomography, to date the most used tool for the diagnosis of lung cancer, have some important limitations, such as the high cost and the need to repeat scanning; moreover, in order to obtain the definitive confirmation of diagnosis, it is almost always necessary to use more invasive diagnostic procedures, for example lung biopsy interventions.

In this context the need arises for identifying alternative diagnostic tools, for example specific and selective markers for lung cancer, which can be easily detected in serum or other biological fluids of patient by means of not invasive procedures, even in an early cancer stage.

To date only few markers are used in the clinical routine. Many others are available, but they are not used in the routine due to their low degree of sensitivity and specificity.

Recent studies have identified the role of the active form of caspase-4 protein as in vitro marker for the diagnosis and/or prognosis of lung cancer.

There are five isoforms of caspase 4: the protein structure of the isoform 1 of the caspase-4, also known as isoform a, consists of three domains; i) CARD (Caspase activation and recruitment domain) domain going from the amino acid (aa) 1 to aa-90, domain responsible for the bond with the portion of lipoteichoic acid of lipopolysaccharide (LPS) (Shi J et al., 2014) through the recognition of transmembrane protein TMEM214 (Shi J et al., 2014); ii) the p20 domain going from aa100-270, catalytic domain or LARGE SUBUNIT, including the active site and the pocket for the substrate; iii) the p10 domain, also defined as SMALL SUBUNIT, going from aa290-377 and including a portion of the pocket of the substrate and the interface for the homodimerization, required to make active the whole enzymatic complex so that the latter carries out its biological activity (FIG. 1). The proteolytic cut which is performed after homodimerization is at the interface between the p20 and p10 domain (FIG. 1A). The pocket of the substrate is characterized by 4 structures (FIG. 1B), identified as S1-4. S1 and S3 (FIG. 1B) are similar and highly preserved between caspases, whereas S4 differs among the various classes of caspases. The S2 portion is highly specific depending upon the caspases taken into consideration and therefore it is characterized by different bond sites for specific substrates by virtue of the biological context.

The isoforms 2-5 of the caspase-4 can derive from a non-sense alternative splicing of the isoform 1. In particular, there are: the isoform p (or isoform 2), with sizes of 36 kDa, consisting of 321 aa and lacking aa 1-56, consisting part of CARD domain of the isoform a (or isoform 1); the isoform 3, lacking aa 117-377, weighing 13 kDa; the isoform 4, lacking aa 264-377 and weighing 30 kDa; and the isoform 5, lacking aa 158-377, weighing 18 kDa.

The European patent application EP3111223A1 describes a method for the diagnosis and/or prognosis of lung cancer comprising the determination or quantification of the sole active form of the human caspase-4 in a sample isolated from a patient. In this patent application no reference was made to the type of isoform of the caspase-4 associated to a positive diagnosis of lung cancer. Although the studies performed up to now have allowed to detect new possible cancer markers and therapeutic targets, however the need remains much felt in the field for detecting more accurate diagnostic markers allowing a correct and quick diagnosis of lung cancer, without the need for having to recourse to biopsy interventions, and which can provide more detailed information about the features and development of the pathology.

### SUMMARY OF THE INVENTION

The inventors have performed several studies aimed at elucidating the role of the human caspase-4 protein as marker for the diagnosis and/or prognosis in vitro of lung cancer.

Thanks to these studies, the authors of the present invention have found that not all isoforms of the human caspase-4 protein are effective markers for the diagnosis of lung cancer: on the contrary, the detection or quantification of the isoform 4 alone allows to obtain a more accurate diagnosis of the pathology.

In particular, the authors of the present invention have surprisingly found that the expression levels of the isoform 4 of the caspase-4 are significantly higher than the levels of the isoform 1 of the caspase-4 inside the tumour mass of the patients affected by lung cancer (FIG. 3A).

An additional feature associated to the expression levels of the isoform 4 of the caspase-4 with respect to the isoform 1 is of prognostic type. In fact, as clearly shown in the experimental section of the present description (FIG. 5), the inventors have found that the higher the levels of isoform 4 are than the levels of isoform 1, the lower the patient survival probability is.

Moreover, the authors of the present invention have also found that the expression levels of the isoform 4 are significantly higher in the blood of sick patients with respect to the blood of healthy subjects or patients with non-tumour respiratory pathologies, by surprisingly finding that the expression degree of such marker allows to discriminate between the benign and malign form of cancer with high sensitivity and specificity.

Therefore, the object of the present invention is to provide methods and kits for a more accurate in vitro diagnosis and/or prognosis of lung cancer, comprising the quantification of the expression levels of the isoform 4 of the caspase-4 in a biological sample obtained from a patient under examination. Such methods provide, in particular, the use of an antibody, devised by the inventors, capable of recognizing a specific epitope of the isoform 4 differing in three amino acids from the isoform 1 of the caspase-4 protein.

Apart from being a support for the formulation of a correct and quick diagnosis, the methods and kits, the invention relates to, allow to perform the diagnosis and/or prognosis directly on plasma samples of the patient under examination, without the need for having to recourse to the use of invasive or particularly expensive diagnostic investigation techniques. Advantageously, the methods and kits, the present invention relates to, could be used even to monitor the development of cancer and to evaluate the effectiveness of a therapy, by easing the adoption of new targeted therapeutic approaches.

In addition to what already illustrated, the authors of the present invention have found that the expression of the “large” sub-unit of the isoform 4 of the human caspase-4 protein is capable of promoting the cancer proliferation. The identification of the role of the isoform 4 of the human caspase-4 protein in lung cancer then has an important relevance even from a therapeutic point of view: the use of compound inhibitors of said isoform 4 has a high potential for the use in the prevention and/or treatment of the pathology, for example by reducing significantly the cancer proliferation.

Therefore, the invention relates to:

an in vitro or ex vivo method for the diagnosis of lung cancer in an individual, including the step of:
a. detecting the expression levels of a first biomarker in a biological sample isolated from said individual, wherein said first biomarker is the isoform 4 of the human caspase-4 protein; an in vitro or ex vivo method to monitor the development of lung cancer in an individual, including the step of:
a. detecting the expression levels of a first biomarker in a biological sample isolated from said individual, wherein said first biomarker is the isoform 4 of the human caspase-4 protein;
   an in vitro or ex vivo method to provide a prognosis on the development of lung cancer in an individual, including the step of:
a. detecting the expression levels of a first biomarker in a biological sample isolated from said individual, wherein said first biomarker is the isoform 4 of the human caspase-4 protein;
   the in vitro use of the isoform 4 of the human caspase-4 protein as biomarker for the diagnosis and/or prognosis and/or to monitor the development of lung cancer;
   an antibody or a fragment thereof capable of specifically binding the isoform 4 of the human caspase-4 protein;
   a kit and use thereof for the diagnosis and/or prognosis and/or to monitor the development of lung cancer, comprising means for detecting the expression levels of the isoform 4 of the human caspase-4 protein in a biological sample, said kit comprising at least an agent for detecting the expression levels of said isoform 4 of the human caspase-4 protein;
   a compound inhibitor of the isoform 4 of the human caspase-4 protein for use in the treatment and/or prevention of lung cancer, wherein said inhibitor is Ac-VVDC-NH₂;
   and an in vitro screening method for the identification of a candidate compound for use in the treatment and/or prevention of lung cancer including the following step:
a. determining the activity of the isoform 4 of the human caspase-4 protein in the presence and in the absence of said candidate compound, where a reduction and/or inhibition of the activity of said isoform-4 indicates that said compound is suitable for use in the treatment and/or prevention of lung cancer.

Other advantages and features of the present invention will result evident from the following detailed description.

### BRIEF DESCRIPTION OF FIGURES

FIG. 1. Known structure of the caspase-4 (isoform 1).

FIG. 2. The isoform 4 of the caspase-4 is present in the tumour mass of patients with NSCLC (A). Representative data of mRNA sequencing of the isoform 4 in the tumour mass (B).

FIG. 3. Protein levels of the isoform 4 of the caspase-4 in the tumour mass of patients with NSCLC (A). ROC analysis (B).

FIG. 4. Higher protein levels than the isoform 4 of the caspase-4 in the blood of patients with NSCLC with respect to healthy, not cancerous, smoking patients and with benign cancers (A). ROC analysis (B).

FIG. 5. Less survival probability in NSCLC patients positive to the isoform 4 of the caspase-4 in the tumour mass.

FIG. 6. The isoform 4 induces cancer proliferation (A) which can be inhibited by the compound 6 (B).

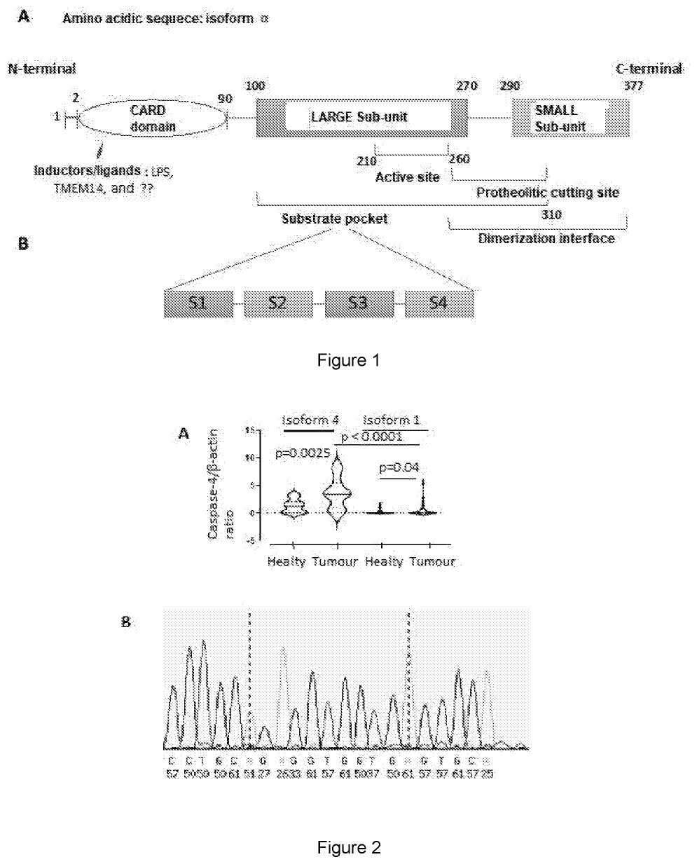

### DETAILED DESCRIPTION OF THE INVENTION

### Glossary

The terms used in the present description are as generally understood by the person skilled in the art, except where differently indicated.

Under the expression “detecting the expression levels” it is meant, in the present description, to detect the presence and/or to quantify the expression levels of the specific isoform of the biomarker, that is of the isoform 4 of the human caspase-4 protein, or, if specified, of the isoform 1 of the human caspase-4 protein.

Under the general term “lung cancer” in the present invention, all forms of lung cancer are indicated which originate from epithelial tissues, belonging to the category of lung cancer, as well as the tumour forms of neuroendocrine origin, such as the lung carcinoid, or of lymphatic origin, such as the lung lymphoma. There are two main types of lung cancer which originate from the epithelial tissue composing the lung structures, which together represent more than 95% of all neoplasms affecting these organs: the small-cell lung cancer, also known as microcytoma, and the non-small-cell lung cancer, often abbreviated as NSCLC from English. Non-small-cell lung cancer in turn can be divided into three main forms: i) spinocellular carcinoma, also known as squamous cell carcinoma or carcinoma with squamous cells; ii) adenocarcinoma and iii) large cell carcinoma.

The term “epitope”, used in the context of the present invention, can be meant as the portion of the isoform 4 of the human caspase-4 protein which is capable of being recognized and bound specifically by an antibody, or antigenic determinant.

The authors of the present invention have surprisingly found that the isoform 4 of the human caspase-4 protein represents an effective biomarker of lung cancer in an individual, and that the detection of the expression levels of said isoform 4 can be used to obtain a diagnosis and/or to monitor the development of lung cancer in a quick and accurate way, and without the need for having to recourse to invasive investigation techniques.

The authors of the invention have further found that it is possible to use the detection of the expression levels of the isoform 4 of the human caspase-4 protein to obtain information of prognostic type about the development of lung cancer in an individual, by allowing to provide accurately the survival probability of a patient affected by such pathology.

The present invention then relates to a method for the diagnosis and/or to monitor the development and/or to provide a prognosis on the development of lung cancer in an individual, including the step of:
a. detecting the expression levels of a first biomarker in a biological sample isolated from said individual, wherein said first biomarker is the isoform 4 of the human caspase-4 protein.

Under the term “isoform 4 of the human caspase-4 protein” the present invention relates to the protein with molecular weight equal to 30 kDa, having an amino acid sequence differing from the canonical sequence, that is the sequence of the isoform 1, or isoform a, of the human caspase-4, (i) due to the absence of amino acids 264-377 aa and (ii) due to the replacement of the amino acid sequence “ANR” at the C-terminal end (fragment in position 261-263 aa) with the sequence “GEC”.

In a more specific way, said isoform 4 of the human caspase-4 protein, in the present description, relates to the human protein identified by the code P49662-4 in the database UniProt, version updated in July 2020, herein reported as SEQ ID. No 1.

According to an aspect of the invention, the herein described method can be used for the diagnosis and/or to monitor the development and/or to provide a prognosis on the development of any one of lung neoplasm typologies of an individual.

According to a preferred aspect, the method, the invention relates to, according to any one of the herein described embodiments could be used for the diagnosis and/or to monitor the development and/or to provide a prognosis on the development of non-small-cell lung cancer (NSCLC).

According to an aspect of the present invention, said step a. of detecting the expression levels of the isoform 4 of the human caspase-4 protein, can be performed on a biological sample isolated from an individual, selected from the group consisting of histological sample, whole blood, plasma or serum. Under histological sample according to the present invention for example a portion or fragment of pulmonary tissue is meant, or in particular a portion or fragment of tumour lung tissue, for example obtained after surgical resection of tumour mass of the lung of an individual, or still a sample of isolated cells. For the preparation of said histological samples any one of the histological techniques, known to the person skilled in the art, could be used.

The term “individual” used in the present description, according to an aspect of the invention, relates to a human being, for example a smoker or an individual subjected to exposure to chemical carcinogenic agents such as asbestos, radon, or heavy metals.

According to an additional aspect of the invention, said individual can be an asymptomatic subject, or a subject who has mild, moderate or severe symptoms. Said individual can also be a subject thereto one or more lung nodules have been diagnosed.

Said individual, according to an additional aspect of the invention, can include a subject who has been subjected to anticancer therapy, for example radiotherapy or chemiotherapy.

According to an aspect of the present invention, said diagnostic and/or prognostic method can include a second step a′ of detecting the expression levels of a second biomarker in a biological sample isolated from said individual, wherein said second biomarker is the isoform 1 of the human caspase-4 protein.

Under the term “isoform 1 of the human caspase-4 protein”, the present invention relates to the canonical isoform of the human caspase-4 protein, having a molecular weight equal to 43.262 kDa, and constituted by three main domains: CARD domain (aa 1-90), p20 domain or “large sub-unit” (aa 100-270), and p10 domain, also defined as “small sub-unit” (aa 290-377). In particular, the present invention relates to the isoform 1 of the human caspase-4 protein identified with the code P49662-1 in the database UniProt version updated in July 2020.

According to a preferred aspect of said invention, said isoform 1 of the human caspase-4 protein has SEQ ID Nr.2.

The method, the present invention relates to, according to any one of the embodiments thereof can also include a further step b. of comparing the expression value determined in step a. with a reference value. Therefore, a detected higher expression value than the reference one will show a negative diagnosis and/or a prognosis, whereas a lower value will show a positive diagnosis and/or prognosis.

According to an aspect of the invention, said reference value can be derived from an analysis performed on a biological sample of an individual affected by a cancer or by an analysis performed on a biological sample of a healthy individual. A possible reference value for example can correspond to the expression level of the isoform 4 of the human caspase-4 protein within a biological sample obtained from an individual affected by lung cancer or by other cancer, or by a healthy individual. Said reference value can also correspond to the expression level of the isoform 1 of the human caspase-4 protein within a biological sample obtained from an individual affected by cancer or from a healthy individual.

According to an aspect of the invention, the method for the diagnosis of lung cancer according to any one of the previously described embodiments, could further include the following steps:
b. comparing the expression value determined in step a. with a reference value obtained from an individual affected by cancer;
c. diagnosing the lung cancer when the value obtained at point a. is higher than or equal to said reference value.

The invention also relates to a method for the diagnosis of lung cancer of an individual, according to any one of the previously described embodiments, further including the following steps:
b. comparing the expression value determined in step a. with a reference value obtained from a healthy individual;
c. diagnosing the lung cancer when the value obtained at point a. is significantly higher than said reference value.

In case of a method to monitor the development of lung cancer, an adequate reference value can be a value corresponding to the expression level of the isoform 4 of the human caspase-4 protein, detected in a biological sample obtained from the same individual in different moments during the therapy and/or during the disease period.

The present invention then also relates to a method to monitor the development of lung cancer, for example to monitor the response of an individual affected by lung cancer to a therapy, according to any one of the herein described embodiments, further including the step of:
b. comparing the value obtained at point a. with a reference value, wherein an increase in the value obtained at point a. with respect to said reference value designates a worsening of the disease and/or a negative response to a therapy, whereas a decrease in the value obtained at point a. with respect to said reference value designates an improvement of the development and/or a regression of the disease, and/or a positive response to a therapy.

The invention also relates to a method for the prognosis of lung cancer of an individual, including the following steps:
a. detecting the expression levels of a first biomarker in a biological sample isolated from said individual, wherein said first biomarker is the isoform 4 of the human caspase-4 protein;
a′. detecting the expression levels of a second biomarker in a biological sample isolated from said individual, wherein said second biomarker is the isoform 1 of the human caspase-4 protein;
b. comparing the value obtained at point a. with the value obtained at point a′, wherein a value obtained at point a. significantly higher than the value obtained at point a′ designates a negative or unfavourable prognosis, and vice versa.

According to an additional aspect of the present invention, said negative or unfavourable prognosis relates to a lower survival possibility.

Under the expression “detecting the expression levels of a first biomarker” according to the present description, “detecting the presence” and/or “quantifying” the expression levels of the isoform 4 of the human caspase-4 protein in a biological sample obtained from an individual, in relative or absolute terms, according to any one of the methods available to the person skilled in the art is meant; according to an aspect of the invention, in the herein described methods the detection and/or quantification of the expression levels of the isoform 4 can be performed by means of the execution of an in vitro test.

Not limiting examples of in vitro tests suitable to be used for the detection of the presence and/or for the quantification of the expression levels of the isoform 4 of the human caspase-4 protein, include, for example, an immunoassay, an aptamer-based assay, a histological or cytological assay, a colorimetric assay, an assay of protein activity, or a mRNA expression level assay.

All types of the above indicated in vitro tests are known to the person skilled in the art who, since he/she knows which isoform of caspase-4 protein has to be detected and/or quantified and on which sample said test has to be performed, could select the most suitable protocol.

An immunoassay suitable for the detection of the presence and/or for the quantification of the expression levels of the isoform 4, according to the present invention, is an immunoassay providing the use of at least an antibody or a fragment thereof capable of specifically binding said isoform 4. An antibody particularly suitable to be used in an immunoassay according to the present invention, is an antibody or a fragment thereof capable of specifically binding said isoform 4 and incapable of binding and/or recognizing any other isoform of the human caspase-4 protein.

Under the expression “incapable of binding and/or recognizing any other isoform of the human caspase-4 protein”, for example, an antibody or a fragment thereof is meant, characterized by a bond affinity for any other isoform of the human caspase-4 protein significantly lower than the bond affinity for the isoform 4 of the human caspase-4 protein. In other words, an antibody suitable to be used in an immunoassay according to the present invention is an antibody or a fragment thereof which in an assay of binding (bond) and/or affinity shows a capability of binding towards any other isoform of the caspase-4 significantly lower or negligible with respect to the capability of binding towards the isoform 4 of the caspase-4.

In a particular embodiment, the detection and/or quantification of the expression levels of the isoform 4 can be performed by means of an immunoassay, by using at least an antibody capable of specifically binding an epitope of the sequence of said isoform 4 of the human caspase-4 protein, including at least the fragment in position 261-263 aa of the C-terminal portion of said isoform-4, corresponding to the amino acid sequence “GEC”. For example, an epitope corresponding to the last 13,12,11,10,9,8,7,6,5,4 3 amino acids of the C-terminal portion of said isoform-4. As previously mentioned, the sequence of three amino acids “GEC” in position 261-263 aa of the sequence of the isoform-4 of the caspase-4 allows to differentiate specifically said isoform with respect to the isoform 1 of the caspase-4, the latter characterized by the amino acid sequence “ANR” in position 261-263 aa.

According to a preferred aspect of the invention, said antibody capable of specifically binding said isoform 4 of the human caspase-4 protein, is an antibody capable of specifically binding an epitope of the sequence of said isoform 4 of the human caspase-4 protein including the fragment 252-263 aa, corresponding to the sequence: VIIVQACRGGEC (SEQ ID Nr. 3).

Said detection and/or quantification of the expression levels of the isoform 4 of the human caspase-4 protein could be performed, for example, by means of an ELISA (Enzyme-Linked Immunosorbent Assay) assay, by using an antibody or a fragment thereof capable of recognizing specifically said isoform 4, in particular an antibody capable of binding said epitope of the sequence of said isoform 4 including the Gly-Glu-Cys sequence, for example the peptide VIIVQACRGGEC (SEQ ID Nr. 3).

An expression assay of the mRNA levels can be performed to check if the isoform 4 is transcribed in said biological sample obtained from an individual. Such assay can be performed, for example, by means of experiments of RT-PCR or “chain reaction of inverse polymerase”, followed by experiments of mRNA sequencing. The extraction of mRNA from said biological sample obtained from an individual can be performed according to any one of the techniques known to the person skilled in the art, for example by using reagents and commercial kits.

The present invention also relates to the use of the isoform 4 of the human caspase-4 protein as biomarker for the diagnosis and/or prognosis and/or to monitor the development of lung cancer, and in particular of non-small-cell lung cancer (NSCL).

The present invention further relates to an antibody or a fragment thereof capable of specifically binding the isoform 4 of the human caspase-4 protein, in particular and a use thereof in the diagnosis and/or monitoring of the development and/or in the prognosis of lung cancer of an individual.

According to an embodiment of the present invention, said antibody is an antibody or a fragment thereof capable of binding an epitope of the C-terminal sequence of said isoform 4 of the human caspase-4 protein including the Gly-Glu-Cys sequence, for example the epitope will correspond to the last 14, 13, 12, 11, 10, 9, 8, 7, 6, 5, 4 amino acids of the SEQ ID nr.1, in a preferred embodiment it will correspond to the fragment VIIVQACRGGEC (SEQ ID Nr. 3). According to a preferred aspect of the invention said antibody is a monoclonal or polyclonal antibody capable of binding an epitope of the C-terminal sequence of said isoform 4 of the human caspase-4 protein, in particular including the fragment VIIVQACRGGEC (SEQ ID Nr. 3).

The antibody, the invention relates to, could be produced according to any one of the conventional techniques known in the field; by purely way of example said antibody could be obtained by immunizing a mouse, or other mammal, against the wished epitope, in particular by using a peptide corresponding to the C-terminal sequence of said isoform 4 of the human caspase-4 protein, said fragment/epitope will correspond for example to the last 14, 13, 12, 11, 10, 9, 8, 7, 6, 5, 4 amino acids of the SEQ ID Nr.1, in a preferred embodiment it will be the peptide VIIVQACRGGEC (SEQ ID No:3). This procedure could be for example performed by injecting the wished epitope in the animal. After having killed the animal, it will be possible to isolate the cells B secreting said antibody against the wished epitope; these isolated cells could be fused in turns with a line of immortalized B cells, of myeloma type, so as to produce a hybridoma cell line, which could be kept in culture for the production of monoclonal antibodies against the wished epitope.

The present invention also relates to the in vitro use of an antibody or a fragment thereof according to any one of the previously described embodiments, in the diagnosis and/or prognosis and/or to monitor the development of lung cancer, in particular in the non-small-cell lung cancer (NSCLC). The invention further relates to a kit and the use thereof for the diagnosis and/or prognosis and/or to monitor the development of lung cancer, comprising means for detecting the expression levels of the isoform 4 of the human caspase-4 protein in a biological sample, said kit comprising at least an agent for detecting the expression levels of said isoform 4 of the human caspase-4 protein.

According to a preferred embodiment, said kit could include at least an antibody or a fragment thereof capable of recognizing, and binding, an epitope of said isoform 4 of the human caspase-4 protein including the fragment VIIVQACRGGEC of the sequence of the protein.

The kit could further include means for detecting the expression levels of said isoform 1 of the human caspase-4 protein in a biological sample, for example an antibody or a fragment thereof capable of binding specifically said isoform 1.

According to an embodiment, said kit could include even means for the detection of the bond between said antibody and said isoform 4 and/or said isoform 1, for example antibodies capable of recognizing said isoform 4 and/or said isoform 1, conjugated to a marker agent.

In an embodiment the kit, the invention relates to, will include also control means which could be used for example to compare the expression levels of the isoform 4 with reference values obtained from the suitable control, for example controls which have known expression levels of said isoform 4 and/or said isoform 1. Such control means includes for example a biological sample obtained from a healthy subject or from a sick subject, wherein the expression level of said isoform 4 and/or said isoform 1 was previously measured.

Such control means can even include one or more agents which are not capable of binding said isoform 4 and/or one or more samples not containing said isoform 4 of the human caspase-4 protein, so as to be able to check the validity of the performed test.

The kit according to any one of the herein described embodiments could further include auxiliary components, such as buffers, fluorophores, chromophores, diluting agents and/or use instructions. Said kit could further include a device for the detection of the expression levels of the isoform 4 of the human caspase-4 protein as described hereinafter.

The detection of the expression levels of the isoform 4 of the human caspase-4 protein for the diagnosis and/or to monitor the development and/or to provide a prognosis on the development of lung cancer in an individual, can be performed even by using a device, for example in form of chip, arranged for such detection, that is capable of recognizing and binding said isoform 4 existing in a biological sample obtained from an individual under examination.

The present invention then also relates to a device for the diagnosis and/or to monitor the development and/or to provide a prognosis on the development of lung cancer in an individual, including at least an agent capable of recognizing or binding, the isoform 4 of the human caspase-4 protein.

According to a preferred aspect of the invention, said at least an agent capable of recognizing, or binding, said isoform 4 of the caspase-4 protein, is an antibody capable of recognizing an epitope of said isoform 4 of the human caspase-4 protein including the fragment VIIVQACRGGEC of the sequence of the protein (SEQ ID Nr. 3).

Said device can include a supporting material on which said agent capable of recognizing, or binding, the isoform 4 of the human caspase-4 protein is immobilized. Not limiting examples of support materials useful to be used for implementing a device according to the present invention include, for example, slides, silicon wafers, paper, cellulose membrane.

The recognition or bond of the isoform 4 of the human caspase-4 protein from/with the immobilized agent could be for example translated and/or amplified in an optical or electric signal.

In an embodiment, the molecules of isoform 4 bound by the immobilized agent could be detected by means of using at least a specific antibody capable of binding said isoform 4, conjugated to a marker agent, for example a fluorophore.

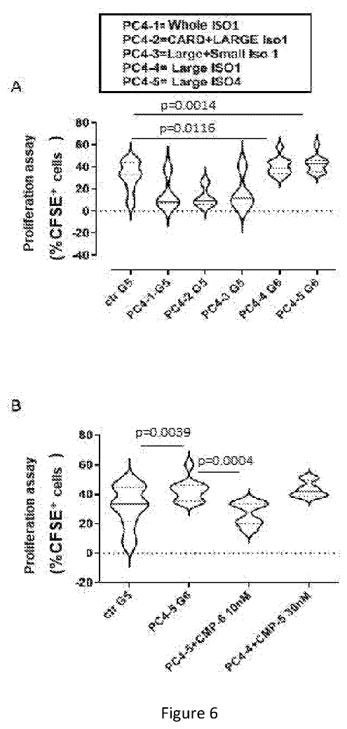

As it is clear in FIG. 6A, the authors of the present invention have found that the expression of the “large” subunit of the isoform 4 of the human caspase-4 protein is capable of promoting the proliferation of cancer cells. After treatment of said cancer cells with the compound Ac-VVDC-NH₂, the inventors have detected a significant decrease in the tumour growth in the cells transfected with the “large” subunit of the isoform 4 (FIG. 6B).

In the light of these experimental observations, the capability of inhibiting said isoform 4 represents an effective strategy for the treatment or the prevention of lung cancer.

The present invention then also relates to a compound inhibitor of the isoform 4 of the human caspase-4 protein for use in the treatment and/or prevention of lung cancer, wherein said inhibitor is Ac-VVDC-NH₂.

According to a preferred aspect of the invention, said compound inhibitor can be used for the treatment and/or prevention of non-small-cell lung cancer.

Said compound inhibitor, or a suitable pharmaceutical composition including said compound inhibitor, could be administered by using any technique comprised in the state of art in the field of oncology, pneumology or the like, to a patient requiring it. Such administration could be performed one or more times, according to the suitable dosages, even based upon the degree of patient response to the therapy, determined for example by monitoring the expression levels of the isoform 4 of the human caspase-4 protein according to any one of the previously described methods.

The present invention also relates to an in vitro method to evaluate and select alternative compound inhibitors of the isoform 4 of the human caspase-4 protein, potentially usable for the prevention and/or treatment of lung cancer.

The present invention then relates to an in vitro screening method for the identification of a candidate compound for use in the treatment and/or prevention of lung cancer including the following step:
a. determining the activity of the isoform 4 of the human caspase-4 protein in the presence and in the absence of said candidate compound, wherein a reduction and/or inhibition of the activity of said isoform-4 indicates that said compound is suitable for use in the treatment and/or prevention of lung cancer.

In an embodiment of the screening method, the invention relates to, said step a. can be performed by means of any one of the techniques known in the art for determining the activity of caspase proteins, in particular of the activity of the human caspase-4 protein.

The present invention also relates to an in vitro screening method for the identification of a candidate compound for use in the treatment and/or prevention of lung cancer including the following steps:
a. treating culture cells with said candidate compound, wherein said cells are lung cancer cells;
b. detecting the expression levels of the isoform 4 of the human caspase-4 protein before and after said treatment step b., wherein a decrease of said expression levels after said treatment step b. indicates that said compound is suitable for use in the treatment and/or prevention of lung cancer.

According to an aspect of the present invention, said step b. of detecting the expression levels of the isoform 4 of the human caspase-4 protein comprises the detection of the presence and/or quantification of the expression levels of said isoform 4 according to any one of the techniques known in the art, and/or according to any one of the previously mentioned techniques.

In a preferred embodiment, said step b. of detecting the expression levels of said isoform 4 can be performed by means of an immunoassay by using an antibody or a fragment thereof capable of binding specifically said isoform 4, in particular an antibody or a fragment thereof capable of binding an epitope of the sequence of said isoform 4 of the human caspase-4 protein including the fragment VIIVQACRGGEC (SEQ ID Nr. 3).

According to a further aspect of the invention, the in vitro screening method according to any one of the previously described embodiments, can include even the following steps:
a. treating culture cells with con said candidate compound, wherein said cells are lung cancer cells;
b. determining and/or quantifying the cancer proliferation levels before and after said treatment step a., wherein a decrease and/or an inhibition of the cancer proliferation after said treatment step a. indicates that said compound is suitable for use in the treatment and/or prevention of lung cancer.

In an embodiment of the screening method according to the present invention, said step c. can be performed by using a fluorochrome capable of evaluating the cell proliferation, by performing an analysis by means of flow cytometry. An example of such fluorochrome is provided by the fluorescent compound “carboxyfluorescein succinimidyl ester” (CFSE), a strongly lipophilic compound capable of binding covalently the amino groups of intracellular macromolecules and which can be kept within the cells. CFSE persists inside the cells allowing a long-term analysis of the marked cells and of the proliferation of the cells taken into consideration.

Herein a method for the prevention and/or treatment of lung cancer is also described, in particular with non-small-cell lung cancer including a step of administering to an individual, requiring it, a compound inhibitor of the isoform 4 of the human caspase-4 protein according to any one of the previously described embodiments.

Such method could include a first step of detecting the expression of one of the herein described biomarkers according to any one of the embodiments and a second step of administering a therapeutic agent for the treatment of said cancer, said second step could provide the administration of one or more drugs suitable for the pathology treatment.

### EXAMPLES

### Materials and Methods

Human samples. Operable lung cancer was diagnosed to patients of NSCLC. Therefore, upon informed consent by the patient, the human samples were obtained after surgical resection during the period 2014-2017. The ethics Committee approval corresponds to number 1254/2014. The age of the healthy not cancerous subjects was 40±10 (average±S.E.M) whereas that of NSCLC patients was 60±10 (average±S.E.M).

RT-PCR and mRNA sequencing. The lung tissues were subjected to the mRNA extraction by using commercial kits (Qiagen, USA) and subjected to inverse retro-transcription to obtain cDNA to be subjected to RT-PCR or sequencing through Sanger method.

ELISA. The presence of the isoform 4 of the caspase-4 was detected by sandwich-like ELISA method obtained by synthetizing a custom antibody based upon the different sequence of the isoform 4 from the isoform 1 (different nucleotide sequence shown in literature: GGTGGTGAGTGCTGA). ELISA test was performed both on lung tissue and on plasma.

Transfections. A549 cells, lung adenocarcinoma, were transfected by means of marketable kits for 72 hours in transient way with the plasmid vectors: 1. PC4-1, miming the whole isoform 1 of the caspase-4; 2. PC4-2, miming CARD+LARGE subunit; 3. PC4-3, miming LARGE+SMALL subunit of the isoform 1; 4. PC4-4, miming LARGE subunit of the isoform 1 and 5. PC4-5, miming LARGE subunit of the isoform 4 of the caspase-4. The cell proliferation was evaluated through method of CFSE (1 μM), analysed by means of flow cytometry. In some experiments, the transfected cells marked with CFSE, were subjected to treatment with two leader compounds (compound 5: COMP-5; compound 6: COMP-6).

Statistical analysis. The statistical analysis of the results was performed through software GraphPad 8.04 (Prism, USA) by One-Way ANOVA method followed by post-test Bonferroni or Mann-Whitney t test, where deemed necessary. The survival curves were analysed by means of Kaplan-Meier method. The data considered to be significant showed a value p<0.05.

Experimental Results

In order to understand if the isoform 4 is transcribed in the lung tumour masses, experiments of RT-PCR (FIG. 2A) were performed, followed by experiments of mRNA sequencing (FIG. 2B). As designated in FIGS. 2A and 2B, such experiments demonstrated that the isoform 4 is expressed in the tumour mass. In particular, the nucleotide sequence differing between the isoform 1 and 4 is the following GGTGGTGAGTGCTGA (SEQ ID Nr.4) the latter existing in the isoform 4 but not in the isoform 1, after an alternative splicing of mRNA between exon 5 and intron 6.

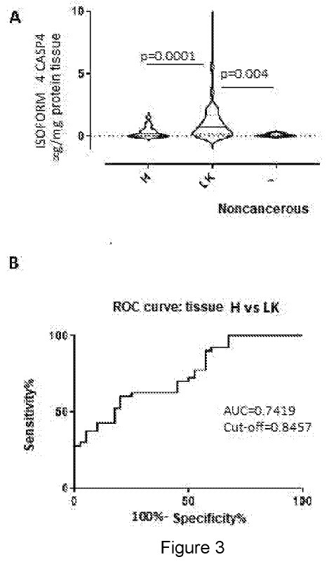

In order to prove the presence of the isoform 4 but not of the isoform 1, a custom antibody was used, capable of recognizing a specific epitope of the isoform 4 differing in 3 amino acids from the isoform 1. Later, a custom ELISA kit was constructed capable of identifying the protein levels of the isoform 4, defined with quantitative method. It was found that the isoform 4 is more expressed than the isoform 1 in the tumour mass of 55 patients out of 85, representing 64.7% of the tested human samples (FIG. 3A).

In particular, the isoform 4 is significantly higher than in the tumour mass, identified as LK (FIG. 3A) with respect to the healthy tissue, identified as healthy (FIG. 3A), and not cancerous (FIG. 3A). Moreover, the ROC analysis highlighted an area underlying the curve (AUC) of 0.7419.

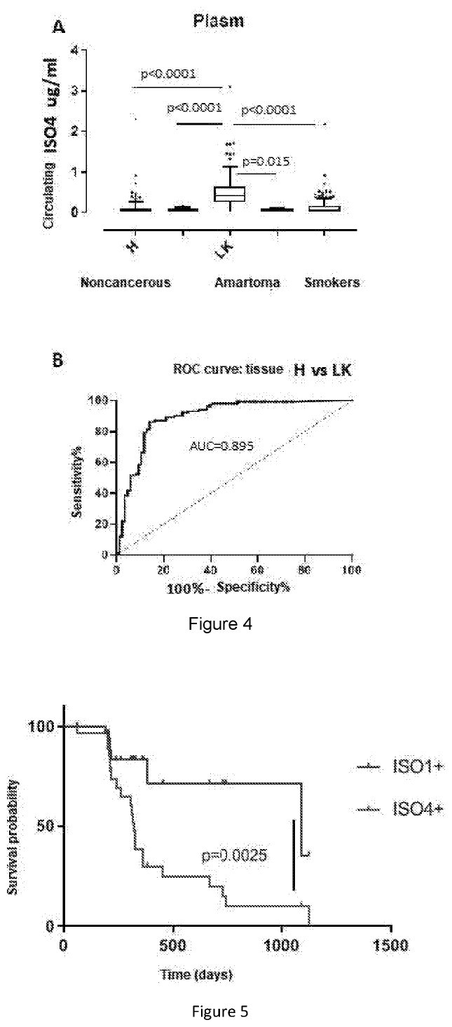

In the same way, the circulating levels of the isoform 4 of the caspase-4 were tested. Such levels were significantly higher in the blood of patients with NSCLC (p<0.0001) with respect to healthy subjects or patients with non-cancerous respiratory pathologies (FIG. 4A, H vs LK). To be noted, the levels of the isoform 4 of the caspase-4 in patients with hamartoma (lung benign cancers), as well as in case of smoking subjects, did not find high levels of such protein (FIG. 4A), by defining the isoform 4 of the caspase-4 the protein capable to discriminate between the cancer benign form and the malignant one. The analysis showed a value of AUC equal to 0.895 (FIG. 4B).

An additional feature associated to the levels of isoform 4 of the caspase-4 is of prognostic type. In fact, it was observed that patients showing higher levels of isoform 4 than the isoform 1 had a lower survival probability (FIG. 5). In particular, the median survival for the patients positive to the isoform 4 is 319 days (10 months) vs 1090 days (36 months) of the patients positive to the isoform 1 (FIG. 5).

In order to understand the role of the isoform 4 in the cancer growth, the A549 cells, cells of lung adenocarcinoma, were transfected with specific vectors, containing specific portions of the isoform 1 and isoform 4 as described in FIG. 6A. It was observed that the isoform 4 and 1 are both capable of promoting cancer proliferation when the LARGE subunit is expressed with respect to the other considered structures (FIG. 6A). At last, the cells transfected with the large subunit of the isoform 1 (PC4-4) and 4 (PC4-5) were treated with leading compound with the purpose of verifying a cancer growth decrease. It was observed that the treatment with the compound 6 (CMP6) at the concentration 10 nM, but not the compound 5 (30 nM), is capable of reducing significantly the cancer growth of the A549 cells transfected with the LARGE portion of the isoform 4 (FIG. 6B).

### LIST OF SEQUENCES IN THE DESCRIPTION

Amino acid sequence of the isoform 4
of the human caspase-4 protein
(https://www.uniprot.org/uniprot/P49662#P49662-4)
SEQ ID No: 1
MAEGNHRKKPLKVLESLGKDFLTGVLDNLVEQNVLNWKEEEKKKYYDAKT
EDKVRVMADSMQEKQRMAGQMLLQTFFNIDQISPNKKAHPNMEAGPPESG
ESTDALKLCPHEEFLRLCKERAEEIYPIKERNNRTRLALIICNTEFDHLP
PRNGADFDITGMKELLEGLDYSVDVEENLTARDMESALRAFATRPEHKSS
DSTFLVLMSHGILEGICGTVHDEKKPDVLLYDTIFQIFNNRNCLSLKDKP
KVIIVQACRGGEC
Amino acid sequence of the isoform 1
of the human caspase-4 protein
(https://www.uniprot.org/uniprot/P49662#P49662-1)
SEQ ID No: 2
MAEGNHRKKPLKVLESLGKDFLTGVLDNLVEQNVLNWKEEEKKKYYDAKT
EDKVRVMADSMQEKQRMAGQMLLQTFFNIDQISPNKKAHPNMEAGPPESG
ESTDALKLCPHEEFLRLCKERAEEIYPIKERNNRTRLALIICNTEFDHLP
PRNGADFDITGMKELLEGLDYSVDVEENLTARDMESALRAFATRPEHKSS
DSTFLVLMSHGILEGICGTVHDEKKPDVLLYDTIFQIFNNRNCLSLKDKP
KVIIVQACRGANRGELWWRDSPASLEVASSQSSENLEEDAVYKTHVEKDF
IAFCSSTPHNVSWRDSTMGSIFITQLITCFQKYSWCCHLEEVFRKVQQSF
ETPRAKAQMPTIERLSMTRYFYLFPGN
Amino acid sequence of epitope of the sequence of
the isoform 4 of the human caspase-4 protein
including the fragment 252-263 aa.
SEQ ID No: 3
VIIVQACRGGEC
Nucleotide sequence differentiating the isoform 4
from the isoform 1 of the human caspase-4 protein
SEQ ID No: 4
GGTGGTGAGTGCTGA

1. An in vitro or ex vivo method for the diagnosis and/or to monitor the development and/or to provide a prognosis on the development of lung cancer in an individual, including the step of:
a. detecting the expression levels of a first biomarker in a biological sample isolated from said individual, wherein said first biomarker is the isoform 4 of the human caspase-4 protein.

2. The method according to claim 1, wherein said lung cancer is non-small-cell lung cancer (NSCLC).

3. The method according to claims 1 or 2, wherein said biological sample is selected from the group consisting of histological sample, whole blood, plasma or serum.

4. The method according to any one or claims 1 to 3, including a further step a′ of detecting the expression levels of a second biomarker in a biological sample isolated from said individual, wherein said second biomarker is the isoform 1 of the human caspase-4 protein.

5. The method according to any one or claims 1 to 4, wherein said detection step comprises the execution of an in vitro test selected from the group consisting of an immunoassay, an aptamer-based assay, a histological or cytological assay or a mRNA expression level assay, in particular wherein at least an antibody or a fragment thereof capable of specifically binding said isoform 4 and not the isoform 1 is used in said immunoassay.

6. An in vitro use of the isoform 4 of the human caspase-4 protein as biomarker for the diagnosis and/or prognosis and/or to monitor the development of lung cancer, in particular of non-small-cell lung cancer (NSCLC).

7. An antibody or a fragment thereof capable of specifically binding the isoform 4 of the human caspase-4 protein and not the isoform 1.

8. An in vitro use of an antibody or a fragment thereof according to claim 7 for use in the diagnosis and/or prognosis and/or to monitor the development of lung cancer, in particular of non-small-cell lung cancer (NSCLC).

9. A kit for the diagnosis and/or prognosis and/or to monitor the development of lung cancer, comprising means for detecting the expression levels of the isoform 4 of the human caspase-4 protein in a biological sample, said kit comprising at least an agent for detecting the expression levels of said isoform 4 of the human caspase-4 protein.

10. The kit according to claim 9, further comprising means for detecting the expression levels of said isoform 1 of the human caspase-4 protein in a biological sample.

11. A compound inhibitor of the isoform 4 of the human caspase-4 protein for use in the treatment and/or prevention of lung cancer, wherein said inhibitor is Ac-VVDC-NH₂.

12. The compound inhibitor according to claim 11, wherein said lung cancer is non-small-cell lung cancer (NSCLC).

13. An in vitro screening method for the identification of a candidate compound for use in the treatment and/or prevention of lung cancer including the following step: determining the activity of the isoform 4 of the human caspase-4 protein in the presence and in the absence of said candidate compound, wherein a reduction and/or inhibition of the activity of said isoform-4 indicates that said compound is suitable for use in the treatment and/or prevention of lung cancer.
